(19) United States
(12) Patent Application Publication
(10) Pub. No.: US 2020/0094021 A1
(43) Pub. Date: Mar. 26, 2020

(54) Padded Forearm Band To Secure Peripherally Inserted Central Catheter (PICC) Lumens, Eden Melmed, US, Spring Lake, NJ

(71) Applicant: Eden Melmed, Spring Lake, NJ (US)

(72) Inventor: Eden Melmed, Spring Lake, NJ (US)

(21) Appl. No.: 16/142,481

(22) Filed: Sep. 26, 2018

(51) Int. Cl. A61M 25/02

(52) U.S. Cl. CPC A61M 25/02; A61M 2025/0266; A61M 2025/024; A61M 2025/0206

(57) ABSTRACT

This invention is meant to address the issues associated with Peripherally Inserted Central Catheter (PICC) lumen. Individuals can have a PICC inserted for extended periods of time. The lumen need to be secured so that they are not hanging freely, whereby they could be pulled or dislodged. The lumen cap and clamp, can cause pain and discomfort when they are pressed into the arm. This can happen from using a PICC cover or simply by resting the arm on a surface. This invention consists of a forearm band with a padded area and a strap. The stretchy forearm band can be pulled on with one hand and positioned with the padded area under the lumen, thereby protecting the arm. The strap can then be pulled over the lumen to secure the lumen in place. This invention can be used alone or under a PICC sleeve and/or shirt sleeve.

### CROSS-REFERENCE TO RELATED APPLICATIONS

Not applicable

### STATEMENT REGARDING FEDERALLY SPONSORED RESEARCH OR DEVELOPMENT

Not applicable

### REFERENCE TO SEQUENCE LISTING, A TABLE, OR A COMPUTER PROGRAM LISTING COMPACT DISC APPENDIX

Not applicable

### BACKGROUND OF THE INVENTION

A Peripherally Inserted Central Catheter (PICC) is a form of intravenous access used for medical treatment. A PICC is used for giving medications, fluids, nutrition and/or drawing blood. It can be used in the hospital setting, nursing facility or at home and can be left in place for weeks to several months as needed.

A PICC is generally inserted into a large vein in the bicep and secured to the bicep with a sterile dressing. The PICC can be a single or double lumen line. A single lumen PICC has one tubing, clamp and cap on the end. A double lumen PICC has two separate tubings, clamps and caps. The lumens are the access points for the PICC and hang outside of the dressing, generally by the inside of the forearm.

The lumens should be protected to avoid the PICC being pulled or dislodged. If not protected, the lumens just hang from the edge of the dressing. At present, protection is generally done with a PICC cover/sleeve that covers mid forearm to upper bicep. These sleeves can be either loose fitting, like a gauze sleeve, or fit like a sock. With the loose fitting sleeve, the lumens have some protection, but are not secured in place. As a result they frequently move and can cause pain and discomfort. With the sock-style sleeve, the lumens are held firmly against the skin and the pressure of the caps and clamps can cause pain and discomfort.

### BRIEF SUMMARY OF THE INVENTION

The discomfort is due to the hard protrusions of the lumen. If using a sleeve, their is no barrier between the lumen and the arm. The clamps in particular do not appear to have been designed with comfort in mind and as such can cause sharp pain in the arm of the individual. The cap can also press into the arm and cause pain. This discomfort can be constant.

The lumens need to be secured. If using a sleeve, it may need to be taken on and off frequently throughout the day as needed for treatment. As one pulls the sleeve on, they must also ensure that the lumens stay in place against the arm and do not get pulled askew or flipped upwards. It is cumbersome for the individual to do this independently because the PICC is often inserted into the right arm, which is the dominant hand for many people. This means single handedly putting the sleeve on and off with their non-dominant hand while also trying to keep the lumens in place.

This invention can be used to address both of these issues; discomfort and securing lumens in place. The invention is a stretchy band worn on the forearm with a padded area and elastic strap to secure the lumens while protecting the forearm from pain. The individual can independently put on and remove the invention easily with one hand and then easily fit the lumens under the elastic strap. The padding between the lumens and the arm will prevent the caps and clamps from pressing into the skin. The invention can also be used under a PICC sleeve and/or shirt sleeve to keep the area clean and/or for privacy.

### BRIEF DESCRIPTION OF THE SEVERAL VIEWS OF THE DRAWING

FIG. 1 is a perspective projection of the invention, including the three parts: forearm band (1), padded area (2) and elastic strap (3).

FIG. 2 is a perspective projection of the invention in use on the forearm. It illustrates the double lumen (4) extending out from the bottom of the sterile dressing (5) and the invention in use both securing the lumen on the forearm band (1) with the strap (3) and protecting the arm with padding (2) between the lumen and the forearm (6).

### DETAILED DESCRIPTION OF THE INVENTION

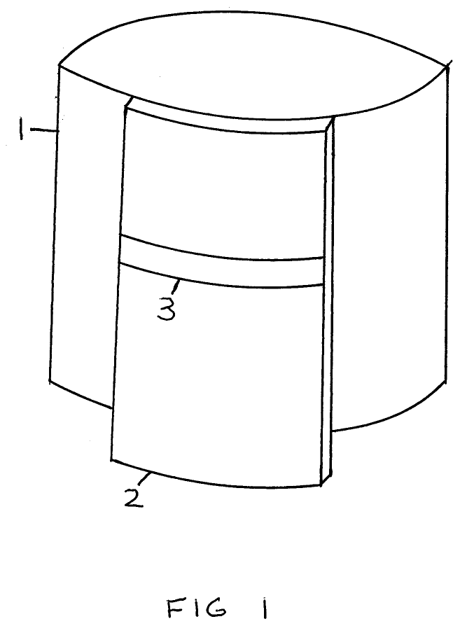

This invention will serve to secure and cushion the PICC lumen. As seen in FIG. 1, the invention has three parts; the forearm band (1), the padded area (2) and the elastic strap (3).

The forearm band is similar to a sweatband that fits on the forearm. It is approximately 3 inches wide and can be made of several different materials that stretch and can easily be pulled on and off with one hand. It should fit comfortably, while not too constricting, and should remain in place until the individual removes it. Different circumferences (sizes) would be needed to accommodate different sized forearms. The thickness of the material should be approximately ⅛- 3/16 inch to allow the invention to be worn under a PICC sleeve and/or shirt sleeve.

The padded area will protect the skin and prevent pain by cushioning the lumens and can be made of several different materials, such as enclosed foam. The padding is a rectangle measuring approximately 3½ inches×2-2¼ inches×⅛- 3/16 inch. This size will easily accommodate two lumens side by side. The lumen cap to clamp measurement is approximately 2¾-3 inches There is variability in this measurement depending on clamp placement on the tubing each time the lumen is accessed. The rectangle should be sewn to the band with a ½ inch overhang on the bottom. Depending on clamp placement, sometimes the clamp can be close to the bend in one's arm. It may not be comfortable or feasible to place the band under the clamp because of the bend in one's arm. This overhang will allow the padding to extend under the clamp in these instances.

The elastic strap should be sewn to the left and to the right sides of the padding approximately 1¼ inch from the top of the band. It should fit around the center of the lumen and hold the lumen in place, but not too tightly.

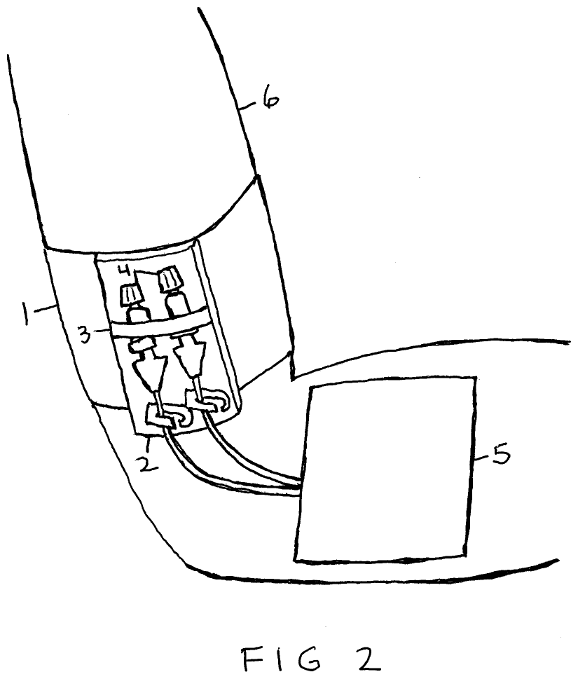

This invention will be used by an individual with a PICC line in the following way. The invention can be used on either arm. Assuming the PICC is inserted into the right bicep, the individual will place the right hand through the band and, using the left hand, will pull the band into position on the forearm. FIG. 2 demonstrates the correct placement and usage of the invention. The padding (2) should be arranged so that the lumen (4) which extend out from the bottom of the sterile dressing (5) will be fully cushioned by it and thus protect the forearm (6). Once the band (1) is in the proper position, the individual can use their left pointer finger to stretch the elastic slightly to allow the lumen to be placed under the strap (3). To remove, the elastic can be lifted slightly to release the lumen and the band pulled off. This invention can be used alone or with a sleeve for privacy and/or to keep the area clean.

The embodiments of the invention in which an exclusive property or privilege is claimed are as follows:

i. A device to both secure PICC lumen and protect the arm consisting essentially of a forearm band, a padded area, and a strap.

ii. The device defined in claim i, wherein the padded area overhangs the bottom of the forearm band.

iii. A device to secure PICC lumen consisting essentially of a forearm band and a strap.

iv. A device to protect the arm from discomfort caused by PICC lumen consisting essentially of a forearm band with padding.

v. The device defined in claim iv, wherein the padded area overhangs the bottom of the forearm band.
